(19) United States
(12) Patent Application Publication
(10) Pub. No.: US 2025/0389726 A1
(43) Pub. Date: Dec. 25, 2025

(54) A QUALITY DETERMINING ARRANGEMENT AND A QUALITY CHECK METHOD

(71) Applicant: DELAVAL HOLDING AB, Tumba (SE)

(72) Inventors: Claus DALLERUP RASMUSSEN, Tumba (SE); Rasmus GRUNDTVIG TAXBØL, Tumba (SE); Lars MOLNIT, Tumba (SE); Ehsan NOROUZI, Tumba (SE)

(21) Appl. No.: 18/879,716

(22) PCT Filed: Jun. 21, 2023
(86) PCT No.: PCT/SE2023/050644

§ 371 Date: Dec. 27, 2024

(30) Foreign Application Priority Data
Jun. 29, 2022 (SE) 2250809-7

(51) Int. Cl. G01N 33/68; B01L 3/00; G01N 21/77; G01N 21/84

(52) U.S. Cl. CPC G01N 33/6842; B01L 3/5023; G01N 21/77; G01N 21/8483; B01L 2200/143; B01L 2300/069; B01L 2300/0825

(57) ABSTRACT

A quality control system for lateral flow sticks detects defective sticks while maintaining them on a carrier. A plurality of lateral flow sticks are arranged at spaced intervals in a Y-direction on the carrier, with each stick oriented transversely and configured to detect biomarkers in milk through capillary flow along a porous membrane with test and control lines. A camera captures images of individual sticks, and a controller analyzes stick quality through image processing to measure parameters like width and position critical for proper flow and test line indication. For sticks determined to be defective, a marker applies a visual indication allowing those sticks to be skipped during subsequent use rather than requiring removal of defective sticks and waste of the entire carrier. The system enables efficient quality control while preserving usability of good lateral flow sticks when defects are found.

### TECHNICAL FIELD

This document discloses an arrangement arranged to determine quality of a lateral flow stick arranged on a carrier comprising a plurality of lateral flow sticks separated from each other on the carrier, which lateral flow sticks are configured for determining a biomarker value of milk extracted from an animal. A corresponding method at a farm, to check quality of lateral flow sticks on a carrier before applying a milk sample to the lateral flow stick for determining a biomarker value of milk extracted from an animal is also disclosed.

### BACKGROUND

On an animal farm, it is important to keep the animals healthy in order to enhance milk/meat production, and/or to monitor when animals are in heat and/or pregnant, for example. It is important to inseminate animals at an optimal moment in order to successfully fertilise the animal. In case the animal is not successfully inseminated, milk production is affected.

Several biomarker measurements may be made on the animal, such as e.g. measuring levels of progesterone, LDH (Lactate Dehydrogenase), BHB (Beta-Hydroxybutyrate) and urea. Thereby important information concerning e.g. heat detection and/or pregnancy of the individual animal may be made (based on measured progesterone level), as well as mastitis (based on LDH) and ketosis (based on BHB). Also, the energy balance of the animal may be estimated (based on urea).

Thereby, a farmer is provided with important information concerning status of each individual animal. However, to perform and analyse biomarker measurements of all individual animals at a farm, e.g. by applying milk samples on prepared dry sticks/lateral flow sticks, and analyse these samples are time consuming for the farmer, who may have to take care of various other important issues. It also put high demands on administrative skills on the farmer to distinguish biomarker measurements from different animals; to keep track on when it is time to repeat the biomarker measurement for each individual animal and when to change biomarker measurement units; maintenance of the biomarker test equipment as well as high demands on cleanliness for not allowing a biomarker measurement of a one animal to be contaminated by biological matters of another animal.

A milk analysis apparatus may be arranged to cooperate with a milk extracting arrangement, for regularly analysing milk samples of the animals, e.g. at or around the moment of a milking session. The milk analysis apparatus may extract a milk sample and provide it on a lateral flow stick/dry stick/lateral flow test strip or other similar milk analysis unit. The milk may typically be diluted with a diluent before being applied to the lateral flow stick.

The lateral flow sticks may be maintained on a carrier, such as e.g. a carrier tape. The carrier tape may in turn be maintained in a cassette as disclosed in document WO 2018236271. Thereby, the lateral flow sticks may be easily administrated at the farm by forwarding one lateral flow stick at the time on the carrier tape. One lateral flow stick is typically used for each test sample. When all the lateral flow sticks on the cassette have been used, the cassette may be replaced for a new one and the old cassette may be returned to the manufacturer for recycling purposes.

The carrier may comprise for example 200 lateral flow sticks or even around 400, for avoiding that the farmer would have to change carrier/cassette too often.

A problem when producing the carrier and applying the lateral flow sticks thereon is that various errors sometimes may occur during the process. An individual lateral flow stick may for example have stains of glue or discolouration, or has been mounted in an unexpected way, for example upside-down, too close to the border of the carrier or another lateral flow stick, etc. The lateral flow stick may have damaged edges or may have been cut too narrow to allow for a reliable biomarker measurement, for example.

These deviations may be detected either immediately during the production or in a quality check after the production, but before allowing the carrier with the lateral flow sticks to reach the user, typically resulting in discarding the whole carrier with all the already mounted lateral flow sticks.

Thus, a carrier comprising one single faulty lateral flow stick currently lead to the waste of all perfectly functional lateral flow sticks on the carrier.

It would be desired to avoid the wasting of lateral flow sticks, thereby saving resources.

### SUMMARY

It is therefore an object of this invention to solve at least some of the above problems and enable usage of a carrier comprising a plurality of lateral flow sticks, also when one or some few lateral flow sticks has/have been disqualified from usage in a quality check.

According to a first aspect of the invention, this objective is achieved by an arrangement arranged to determine quality of a lateral flow stick arranged on a carrier comprising a plurality of lateral flow sticks. The arrangement is typically installed at a production site where the lateral flow sticks are mounted on the carrier.

The carrier has a longitudinal extension in a Y-direction. The lateral flow sticks are individually arranged on the carrier and separated from each other at a distance from each other in the Y-direction. An elongated direction of each lateral flow stick extends in a substantially transverse direction in relation to the Y-direction of the carrier, for example with about 45-90 degrees rotational dislocation in relation to the Y-direction.

The lateral flow sticks are configured for determining a biomarker value of milk extracted from an animal.

A biomarker, or biological marker, generally refers to a measurable indicator of some biological state or condition of the animal. The biomarker value measurement may be associated with pregnancy/reproduction of the animal, health of the animal, and/or quality of the milk of the animal.

The arrangement comprises a camera directed to capture an image of at least one of the lateral flow sticks out of the plurality of lateral flow sticks, after having been arranged, i.e. mounted, on the carrier. The arrangement also comprises a marker, arranged to apply a visual indication on the lateral flow stick, or at the carrier, indicating a quality of that lateral flow stick. In addition, the arrangement also comprises a controller, communicatively connected to the camera and to the marker. The controller is configured to obtain an image of the at least one lateral flow stick and part of the carrier, from the camera. The controller is also configured to perform image processing of the obtained image. In addition the controller is configured to determine quality of the at least one lateral flow stick based on the performed image processing. The controller is furthermore configured to generate a command to the marker, to apply the visual indication indicating quality of the at least one lateral flow stick.

The visual indication of the marker may for example comprise an integer, a letter, a geometric shape, a colour, a string of letters, a bar code, a QR code, a punched hole in the carrier, etc.

The quality check of each individual lateral flow stick on the carrier is made during production or after production but before distribution of the carrier with the lateral flow sticks to the respective user's farm. When any lateral flow stick is detected by image processing, which does not fulfil certain predetermined criteria such as e.g. shape of the lateral flow stick, width of the lateral flow stick, colour of the lateral flow stick, position of the lateral flow stick, etc., that lateral flow stick is marked by the visual indication.

Thanks to the visual indication marking the faulty lateral flow stick, the carrier may be provided anyway to the user. At the user's farm, upon detection of the visual indication, the carrier is forwarded, passing the indicated/marked lateral flow stick to a subsequently following lateral flow stick on the carrier, to which the test could be made.

By adding a number of additional lateral flow sticks to the carrier, the user is compensated for the faulty lateral flow stick that could not be used.

By the interplay between the arrangement for detection and marking of any faulty lateral flow stick before distribution, and the detection of the visual indication at the user side, the requirements of the controller hardware at the user's facility could be kept very low, as no quality check has to be made at the farm; only a monitoring for the presence/absence of the visual indication.

Thanks to the provided solution, the carrier does not have to be wasted just because one single, or some few faulty lateral flow sticks on the carrier, which saves resources. Yet, the user is assured that he/she receives at least as many lateral flow sticks on the carrier that he/she is expecting; and that the biomarker measurements of the lateral flow sticks that has passed the quality check of the manufacturer could be relied upon.

In a first implementation of the arrangement according to the first aspect, the controller may be configured to perform the image processing and estimate width of the at least one lateral flow stick; and wherein the quality may be related to the estimated width of the at least one lateral flow stick. Thus, a comparison may be made between the estimated width of the lateral flow stick with a minimum reference width. Any lateral flow stick having a width not exceeding the minimum reference width may be considered having an insufficient quality and be disqualified from usage.

In case the lateral flow stick has a width which is narrower than the minimum reference width, a lateral flow test may not be performed for several reasons. Firstly, it may be difficult or even impossible to find the lateral flow stick, and/or to align the needle with the very narrow lateral flow stick. Another reason may be that the amount of reactive particles on the lateral flow stick may be too few to provide a reliable visual indication on a test line and or control line of the lateral flow stick.

By disqualifying lateral flow sticks that are too narrow, i.e. has a width narrower than the minimum reference width, the risks of failed lateral flow tests for this reason at the user side is eliminated, or at least reduced.

In a second implementation of the arrangement according to the first aspect, or any implementation thereof, the controller may be configured to perform the image processing by contour analysis of the at least one lateral flow stick in the image. The controller may also be configured to determine an outer contour of the at least one lateral flow stick based on a contour analysis. The controller may additionally be configured to compare the determined outer contour of the at least one lateral flow stick with a reference contour. The quality may be related to a difference between the determined outer contour of the at least one lateral flow stick and the reference contour. Thus, a lateral flow stick having a determined outer contour deviating from the reference contour may be considered having an insufficient quality and be disqualified from usage.

In case the lateral flow stick has an outer contour which is deviating from the reference contour, e.g. due to a damaged edge, an asymmetric cut of the lateral flow stick or the like, the lateral flow test may not be successfully performed at the farm.

By disqualifying lateral flow sticks that deviates from the reference contour, the risks of failed lateral flow tests for this reason at the user side is eliminated, or at least reduced.

In a third implementation of the arrangement according to the first aspect, or any implementation thereof, the controller may be configured to, based on the image processing: detect discolouration on the at least one lateral flow stick; and wherein the quality may be related to colour and/or discolouration of the at least one lateral flow stick. A lateral flow stick having a colour and/or discolouration deviating more than a threshold level from a reference colour may be considered having an insufficient quality and be disqualified from usage.

In case the lateral flow stick has a colour deviating from the expected reference colour, irrespectively of reason, it may be a sign of insufficient quality for successful usage.

By disqualifying lateral flow sticks that deviates from the expected reference colour, the risks of failed lateral flow tests for this reason at the user side is eliminated, or at least reduced.

In a fourth implementation of the arrangement according to the first aspect, or any implementation thereof, the controller may be configured to, based on the image processing: determine whether the at least one lateral flow stick is arranged on the carrier in a predetermined way. The quality may be related to the arrangement of the at least one lateral flow stick on the carrier in comparison with the predetermined way of arrangement.

In case the lateral flow stick is arranged on the carrier for example upside down, in the reversed direction, with an unexpected inclination outside a reference interval, twisted, gathered in a cluster, etc., it will likely be impossible to determine the biomarker value of the milk sample at the farm with the lateral flow stick in question.

By disqualifying lateral flow sticks that deviates from the expected way of arrangement on the carrier, the risks of failed lateral flow tests for this reason at the user side is eliminated, or at least reduced.

In a fifth implementation of the arrangement according to the first aspect, or any implementation thereof, wherein shape of the visual indication, number of visual indications and/or position of the visual indications at the carrier or the lateral flow stick as provided by the marker, forms a carrier of information concerning the determined quality of the lateral flow stick.

Thanks to the provided visual indication, various information could be provided to the arrangement of the farm, concerning the lateral flow stick. The provided information may for example be a rating of the estimated quality of the lateral flow stick (lateral flow sticks of different quality levels may be used for different purposes); an instruction to disallow the lateral flow stick; an instruction to forward the carrier to the subsequent lateral flow stick; an instruction to adjust the speed when forwarding the carrier to the subsequent lateral flow stick; etc.

In a sixth implementation of the arrangement according to the first aspect, or any implementation thereof, the marker may be arranged to apply the visual indication by punching a hole in the carrier.

An advantage by providing the visual indication as a hole in the carrier is that the marking becomes very easily recognisable at the user side.

In a seventh implementation of the arrangement according to the first aspect, or any implementation thereof, the marker may be arranged to apply the visual indication on a predetermined position on the carrier.

By providing the visual indication at a predetermined position on the carrier, which predetermined position is dedicated for a particular lateral flow stick, the quality of the lateral flow stick in question may easily and unambiguously be determined at the user side/farm.

In an eighth implementation of the arrangement according to the first aspect, or any implementation thereof, the predetermined position for the visual indication may be situated on the carrier in close vicinity of or associated with the lateral flow stick to which the quality is determined.

In a ninth implementation of the arrangement according to the first aspect, or any implementation thereof, the carrier may be arranged to be forwarded in the Y-direction, thereby sequentially placing one lateral flow stick at the time in a position where the camera is enabled to capture the image, when the image processing is to be performed by the controller; wherein the predetermined position for the visual indication of the lateral flow stick is situated on the carrier in close vicinity of a previous lateral flow stick, in the Y-direction of the carrier.

When the visual indication is detected at the user side, which may be made when taking a photo of a lateral flow stick n, that visual indication is associated with the subsequent lateral flow stick n+1. The quality of the subsequent lateral flow stick n+1 could thereby be immediately determined. In case the visual indication of the subsequent lateral flow stick n+1 shows that the lateral flow stick in question is disqualified from usage, the carrier could be forwarded immediately to the next subsequent lateral flow stick n+2, for repeating the procedure there.

According to a second aspect of the invention, this objective is achieved by a method at a farm. The method aims at performing an action in relation to a lateral flow stick on a carrier comprising a plurality of lateral flow sticks, based on the quality of that lateral flow stick as indicated by a visual indication that has been provided to the carrier and/or the lateral flow stick by an arrangement according to the first aspect, or any implementation thereof.

The method comprises the step of obtaining an image of a part of the carrier and/or the lateral flow stick. The method also comprises the step of performing image processing of the obtained image. In addition, the method also comprises the step of detecting the visual indication on the lateral flow stick, or on the carrier, indicating a quality of that lateral flow stick. The method also comprises the step of performing an action in relation to the lateral flow stick, based on the quality of that lateral flow stick as indicated by the detected visual indication.

Thanks to the visual indication marking the faulty lateral flow stick, it could directly and unambiguously be determined at the farm whether the lateral flow stick has a quality allowing for application of a lateral flow test or not, for determining the biomarker value of the applied milk sample, without having to make any own independent quality check of the lateral flow sticks on the carrier. Thus, the user is assured that all lateral flow tests made on any approved lateral flow stick, i.e. any lateral flow stick associated with a visual indication of a quality indicating approval by the manufacturer could reliably be used for biomarker measurements, without having to make any own independent quality check of the lateral flow stick.

In a first implementation of the method according to the second aspect, the action to be performed in relation to the lateral flow stick may comprise applying a milk sample of milk extracted from an animal to the lateral flow stick when the detected visual indication of that lateral flow stick allow the lateral flow stick to be used for determining a biomarker value of the milk sample.

In a second implementation of the method according to the second aspect, or any implementation thereof, the action to be performed in relation to the lateral flow stick may comprise disallowing application of a milk sample of milk extracted from an animal to the lateral flow stick when the detected visual indication of that lateral flow stick does not allow the lateral flow stick to be used for determining a biomarker value of the milk sample.

In a third implementation of the method according to the second aspect, or any implementation thereof, the carrier may have a longitudinal extension in a Y-direction and be arranged to be forwarded in the Y-direction, thereby sequentially placing one lateral flow stick at the time in a position where the application of the milk sample is enabled. Each one of the lateral flow sticks may be individually arranged, separated with a minimum distance from any other lateral flow stick on the carrier.

The action to be performed in relation to the lateral flow stick when the detected visual indication of that lateral flow stick does not allow the lateral flow stick to be used for determining the biomarker value comprises forwarding the carrier in the Y-direction to a subsequent lateral flow stick for repeating the method steps of the second aspect, or any implementation thereof, on the subsequent lateral flow stick.

In a fourth implementation of the method according to the second aspect, or any implementation thereof, the action to be performed in relation to the lateral flow stick when the detected visual indication of that lateral flow stick does not allow the lateral flow stick to be used for determining the biomarker value, may comprise adjusting the speed when forwarding the carrier in the Y-direction to the subsequent lateral flow stick.

In some cases, for example when the lateral flow stick has spots of glue on it, or when two or more lateral flow sticks have been glued onto each other on the carrier, it is desired to forward the carrier with reduced speed, i.e. a speed lower than a threshold limit. Problems related to the forwarding of the carrier is thereby avoided, or at least minimised.

In a fifth implementation of the method according to the second aspect, or any implementation thereof, the image may be obtained from a part of the carrier comprising a predetermined position dedicated for the visual indication indicating quality of one particular lateral flow stick.

Thereby, the quality of the lateral flow stick is obtained by checking just the predetermined position on the dedicated part of the carrier, which is reducing search time for the visual indication. The risk of, by mistake, interpret for example an accidental spot or discolouration on the carrier for a visual indication is also radically reduced.

Yet an advantage with using the predetermined position on the dedicated part of the carrier for transferring the quality information of the lateral flow stick is that also absence of a visual indicator at the predetermined position could advantageously be used as quality indicator of the lateral flow stick. For example, no visual indicator at all at the predetermined position on the dedicated part of the carrier indicates premium quality of the lateral flow stick.

By the automated recognition of faulty lateral flow sticks according to the disclosed method, the requirements for intervention and/or manual input by the farmer is eliminated or at least reduced. Costs, maintenance and work intensity of the farmer associated with management of lateral flow sticks for determining biomarker values of milk samples are thereby also reduced.

Other advantages and additional novel features will become apparent from the subsequent detailed description.

### FIGURES

Embodiments of the invention will now be described in further detail with reference to the accompanying figures, in which:

FIG. 1 illustrates an example of a production site arrangement arranged to monitor production of a carrier comprising a plurality of lateral flow sticks for determining a biomarker value of milk extracted from an animal.

FIG. 2 illustrates an image of a carrier with a lateral flow stick, captured from above by a camera, according to an embodiment.

FIG. 3 illustrates an example of an arrangement at a farm, arranged to monitor production of a carrier tape comprising a plurality of lateral flow sticks to perform an action in relation to a lateral flow stick on a carrier.

FIG. 4 schematically illustrates an embodiment of a method at a farm, to perform an action in relation to a lateral flow stick on a carrier.

### DETAILED DESCRIPTION

Embodiments of the invention described herein are defined as an arrangement and a method at a farm, which may be put into practice in the embodiments described below. These embodiments may, however, be exemplified and realised in many different forms and are not to be limited to the examples set forth herein; rather, these illustrative examples of embodiments are provided so that this disclosure will be thorough and complete.

Still other objects and features may become apparent from the following detailed description, considered in conjunction with the accompanying drawings. It is to be understood, however, that the drawings are designed solely for purposes of illustration and not as a definition of the limits of the herein disclosed embodiments, for which reference is to be made to the appended claims. Further, the drawings are not necessarily drawn to scale and, unless otherwise indicated, they are merely intended to conceptually illustrate the structures and procedures described herein.

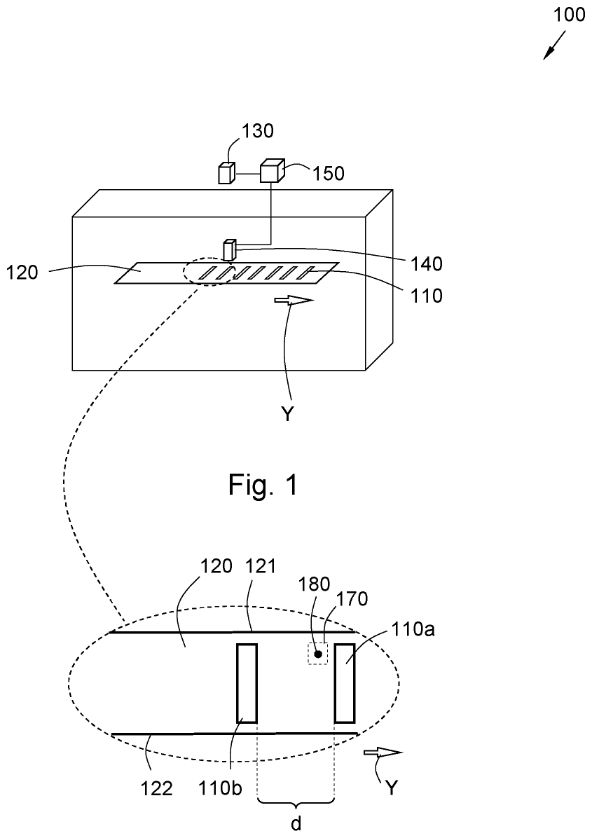

FIG. 1 illustrates a scenario with a production site arrangement 100 arranged to determine quality of lateral flow sticks 110a, 110b, 110c arranged on a carrier 120, and provide a visual indication on either the respective lateral flow stick 110a, 110b, 110c, or on the carrier 120, concerning the determined quality.

The arrangement 100 comprises a camera 130 configured for and directed to capture an image of one lateral flow stick 110b on the carrier 120 at the time. The arrangement 100 also comprises a marker 140, arranged to apply the visual indication 180 on the lateral flow stick 110b, or at the carrier 120 associated with the lateral flow stick 110b, indicating the quality of that lateral flow stick 110b.

The quality of the lateral flow stick 110b is determined by a controller 150 comprised in the arrangement 100. The controller 150 is communicatively connected to the camera 130 and to the marker 140.

The controller 150 is configured to determine quality of the lateral flow sticks 110a, 110b, 110c on the carrier 120 based on image processing of a respective image depicting one of the lateral flow sticks 110a, 110b, 110c, captured by the camera 130. Quality of the lateral flow stick 110a, 110b, 110c in this context may be estimated on a binary scale, i.e. allowed/disallowed for usage; or be estimated on a graded scale with for example 5 levels (non-limiting arbitrary example).

The controller 150 is configured to obtain an image of the at least one lateral flow stick 110b and part of the carrier 120 from the camera 130, for example by triggering a photographic action by the camera 130, to capture the image of the lateral flow stick 110b and provide it to the controller 150.

The controller 150 is also configured to perform image processing of the obtained image.

The image processing concerns an analysis of one or several predetermined critical parameters of the lateral flow stick 110b, for example width of the lateral flow stick 110b, position of the lateral flow stick 110b on the carrier 120, colour of the lateral flow stick 110b, outer contour of the lateral flow stick 110b, thickness of the lateral flow stick 110b, etc. A visual error check of the lateral flow stick 110b is thereby made.

Based on the performed image processing, the quality of the lateral flow stick 110b is determined by the controller 150, for example by comparing the determined width of the lateral flow stick 110b with a reference minimum width of the lateral flow sticks, and disqualifying too narrow lateral flow sticks 110a, 110b, 110c.

Having determined the quality of the lateral flow stick 110b, a command is generated and sent to the marker 140, to apply the visual indication 180 indicating quality of the lateral flow stick 110b.

The visual indication 180 concerning the quality of the lateral flow stick 110b may be applied on a predetermined position 170 on the carrier 120 in some embodiments, for example by punching a hole in the carrier 120 on the predetermined position 170. The visual indication 180 may however comprise a colour mark, a graphical shape, a letter, a number, a code such as a QR code, bar code, morse code, etc.

Thus, the shape of the visual indication 180, the number of visual indications 180 and/or position of the visual indications 180 on the carrier 120 or on the lateral flow stick 110b, may form a carrier of information concerning the determined quality of the lateral flow stick 110b.

One particular visual indication 180 may mark a lateral flow stick 110a, 110b, 110c with approved quality and another distinct visual indication 180 may mark a lateral flow stick 110a, 110b, 110c with a disapproved quality. In some embodiments, the visual indication 180 may be made only for disapproved lateral flow sticks 110a, 110b, 110c, i.e. no visual indication 180 may be provided when the lateral flow stick 110a, 110b, 110c has an approved quality; or alternatively vice versa, only for approved lateral flow sticks 110a, 110b, 110c.

The predetermined position 170 for the visual indication 180 may be situated on the carrier 120 in close vicinity the lateral flow stick 110b to which the quality is determined in some embodiments.

The carrier 120, for example embodied as a carrier tape, a blister or similar, is extending in a Y-direction. The plurality of lateral flow sticks 110a, 110b, 110c comprised on the carrier 120 are separated from each other and situated at a distance d from each other on the carrier 120. The lateral flow sticks 110a, 110b, 110c may also be situated with a minimum margin distance to any lateral edge 121, 122 of the carrier 120.

The lateral flow sticks 110a, 110b, 110c are arranged on the carrier 120 so that an elongated direction of each lateral flow stick 110a, 110b, 110c extends in a substantially transverse direction in relation to the Y-direction. The lateral flow sticks 110a, 110b, 110c may for example be arranged with a 45-90-degree deviation from the Y-direction.

Each one of the lateral flow sticks 110a, 110b, 110c is configured for determining a biomarker value of milk extracted from an animal at a farm.

“Animal” may be any arbitrary type of domesticated female milk producing mammal such as cow, goat, sheep, etc., typically kept at a dairy farm.

The biomarker to be determined by the lateral flow stick 110a, 110b, 110c may be e.g. progesterone, glycoprotein, oestrogen and/or Gonadatropin-Releasing Hormones, or any other similar biomarker associated with reproduction, milk production or general health of the animal, in different embodiments.

The lateral flow stick 110a, 110b, 110c, may also be referred to as dry stick, lateral flow test strip, milk analysis unit, etc. The biomarker value of extracted milk may e.g. indicate progesterone in a milk sample by a lateral flow test, when the milk sample is applied to the lateral flow stick 110a, 110b, 110c.

The production site arrangement 100 may be situated at a production site where the lateral flow sticks 110a, 110b, 110c are mounted onto the carrier 120, before the carrier 120 with the lateral flow sticks 110a, 110b, 110c is distributed to a dairy farm.

The arrangement 100 may also comprise one or several motors for forwarding the carrier 120 in the Y-direction, thereby enabling the camera 130 to capture an image of one lateral flow stick 110a, 110b, 110c at the time, for the image processing purposes. The camera 130 may thereby be enabled to capture the image of the lateral flow stick 110a, 110b, 110c when the image processing is to be performed by the controller 150.

The carrier 120 may in some embodiments, but not necessarily, be comprised in a cassette or roll.

The carrier 120 (or the cassette or roll comprising the carrier 120) may be detachably inserted in the arrangement 100 after or during mounting of the lateral flow sticks 110a, 110b, 110c on the carrier 120.

In some embodiments, the carrier 120 may be arranged to be forwarded in the Y-direction, thereby sequentially placing one lateral flow stick 110b at the time in a position where the camera 130 is enabled to capture the image of the lateral flow stick 110b to which the quality is determined by the controller 150. The predetermined position 170 for the visual indication 180 of the lateral flow stick 110b to which the quality is to be determined may be situated in close vicinity of a previous lateral flow stick 110a in the Y-direction.

An advantage therewith, when the carrier 120 has been distributed to the farm, is that when a camera of the farm captures an image of the previous lateral flow stick 110a in the Y-direction, it is thereby known if the next/subsequent lateral flow stick 110b on the carrier 120 in the Y-direction has a quality allowing for a lateral flow test or not. The forwarding speed of the carrier 120 may be determined based on the quality of the next/subsequent lateral flow stick 110b.

Some examples of possible errors, i.e. deviations from an expected state, of the lateral flow stick 110b that the controller 150 may detect when performing the image processing, or visual error check procedure, may be e.g. to detect glue, dirt or discolouration on the lateral flow stick 110b; detect that the position of the lateral flow stick 110b is at least partly outside an expected position range of the lateral flow stick 110b on the carrier 120; detect that there is no lateral flow stick 110b in the expected position range of the lateral flow stick 110b on the carrier 120; detect that the lateral flow stick 110b is mounted upside-down on the carrier 120; and/or detect that the lateral flow stick 110b is mounted in reversed direc-tion on the carrier 120.

It hereby becomes possible to utilise the carrier 120 at a farm, also when some few lateral flow sticks 110a, 110b, 110c are not possible to use.

It is thereby avoided that carriers 120/cassettes comprising the carrier 120 are wasted, just because one or some few lateral flow sticks 110a, 110b, 110c on the carrier 120 is/are defect.

At the farm, the visual indication 180 identifying the faulty lateral flow stick 110b is considerably easier to detect (i.e. requiring less computational resources) than in case the image processing of the image lateral flow stick 110b comprising determine quality of the at least one lateral flow stick 110b based on the performed image processing would have to be performed at each farm.

Resources are saved, as less carriers 120/cassettes comprising carriers 120 have to be wasted, but also as the computational efforts of the farm site controller are saved.

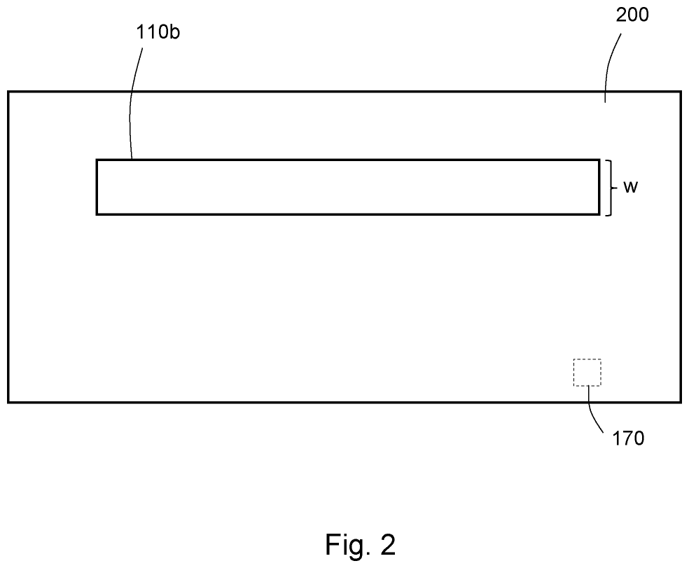

FIG. 2 illustrates an example of an image 200 of a lateral flow stick 110b on a carrier 120, captured by a camera 130 of an arrangement 100 as illustrated in FIG. 1, or a camera comprised in a corresponding arrangement at a farm.

The image 200 is provided to a controller 130 of the arrangement 100 for performing image processing of the obtained image 200. Hereby, the quality of the at least one lateral flow stick 110b based on the performed image processing is determined by the controller 130.

The width w of the lateral flow stick 110b may be estimated during the image processing in some embodiments. The quality of the lateral flow stick 110b may be related to the estimated width w of the lateral flow stick 110b. A comparison may be made with a reference minimum width of the lateral flow sticks. In case the lateral flow stick 110b is narrower than the reference minimum width, the lateral flow stick 110b may be disqualified.

An example of a predetermined position 170 for the visual indication 180 indicating the determined quality of the lateral flow stick 110b is also illustrated in the image 200.

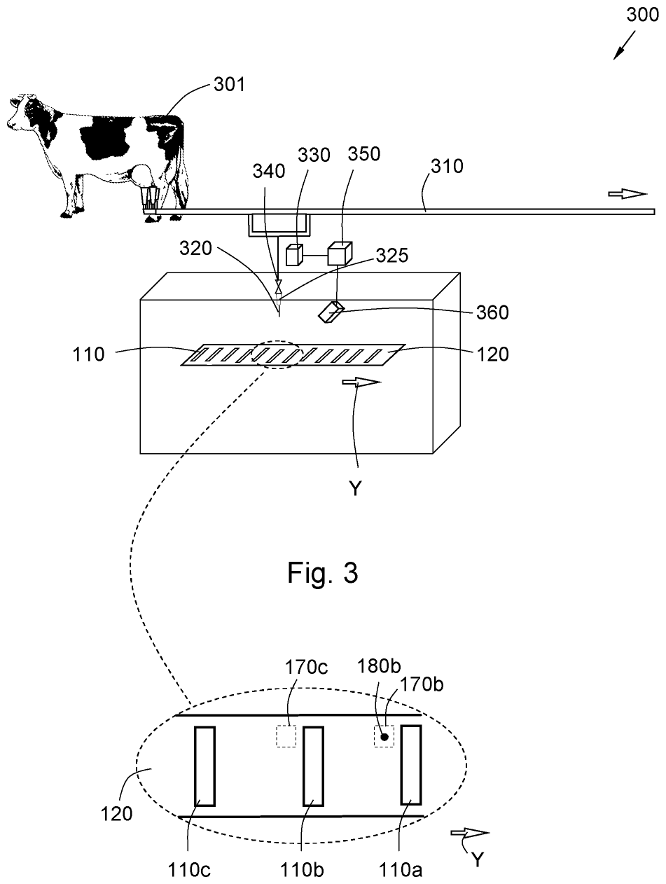

FIG. 3 illustrates an arrangement 300 at a farm. Milk of an animal 301 may be extracted and evacuated by a milking equipment that are part of a milking parlour via a milk line to a milk tank or similar milk storage.

A milk sample of the animal 301 may be extracted during a milking session and be provided via a valve 340 to a tube element 325, configured to receive the milk sample of the animal 301 and provide the milk sample to a needle 320.

The arrangement 300 comprises various electronics and equipment such as a camera 330, one or several pumps, the tube element 325, the needle 320, a controller 350, possibly a light source 360. The arrangement 300 may also comprise a motor or other similar device for forwarding the carrier 120 in a Y-direction.

The intention is to provide the milk sample via the needle 320 to a lateral flow stick 110b on a carrier 120.

However, firstly a method is performed, to determine whether the quality of the lateral flow stick 110b allows for application of the milk sample.

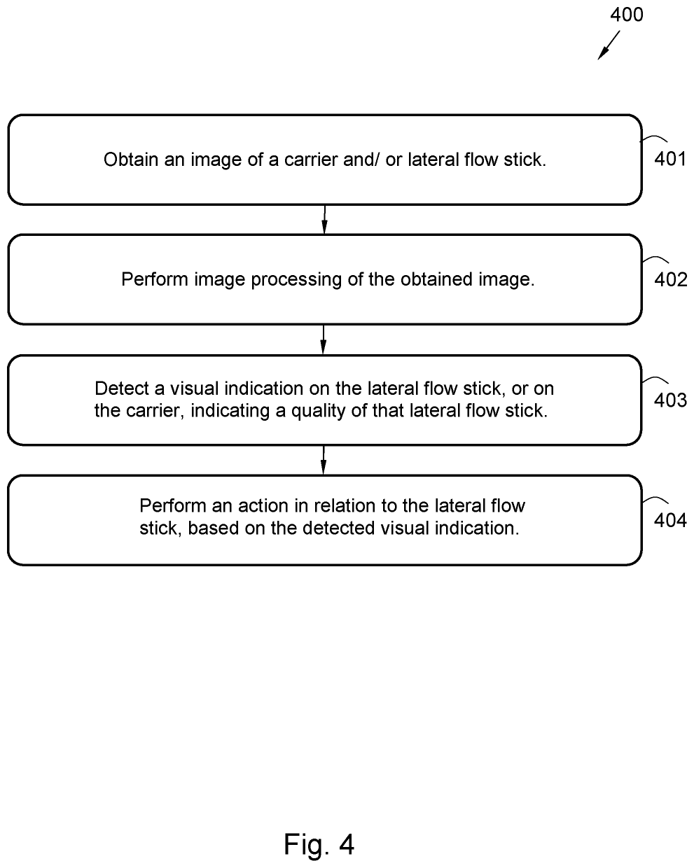

The method 400 is illustrated in FIG. 4. The method 400 may be performed by a controller 350 of an arrangement 300 at a farm. The method 400 aims at performing an action in relation to a lateral flow stick 110b on a carrier 120 comprising a plurality of lateral flow sticks 110a, 110b, 110c. The carrier 120 may have longitudinal extension in a Y-direction and be arranged to be forwarded in the Y-direction, thereby sequentially placing one lateral flow stick 110a, 110b, 110c in a position where the application of the milk sample is enabled.

Each one of the lateral flow sticks 110a, 110b, 110c is individually arranged, separated with a minimum distance from any other lateral flow stick 110a, 110b, 110c on the carrier 120.

The action to be performed is based on the quality of the lateral flow stick 110b in question, as indicated by a visual indication 180 that has been provided to the carrier 120 and/or the lateral flow stick 110b by the previously described arrangement 100, illustrated in FIG. 1.

The visual indication 180 is a visual marker such as e.g. an integer, a letter, a geometric shape, a colour, a string of letters, a hole in the carrier 120 and/or the lateral flow sticks 110a, 110b, 110c, an encoding such as a bar code, QR code, etc., which may carry information concerning quality of the associated lateral flow stick 110a, 110b, 110c.

The method 400 comprises obtaining 401 an image 200 of a part of the carrier 120 and/or the lateral flow stick 110b. The image 200 may be captured by a camera 330 of the arrangement 300, possibly triggered by the controller 350 of the arrangement 300.

The image 200 may be obtained 401 from a part of the carrier 120 comprising a predetermined position 170, dedicated for the visual indication 180 indicating quality of one particular lateral flow stick 110b.

The method 400 also comprises performing 402 image processing of the obtained 401 image 200.

Image processing or computer vision is a technical field comprising methodology for acquiring, processing, analysing, and understanding images and, in general, high-dimensional data from the real world in order to produce numerical or symbolic information.

In this case, the computer vision comprises searching for a visual indication 180 on the lateral flow stick 110b, or on the carrier 120, indicating a quality of the lateral flow stick 110b.

The method 400 also comprises detecting 403 the visual indication 180 on the lateral flow stick 110b, or on the carrier 120. When detecting the visual indication 180, it may be decoded by the controller 350, thereby enabling determination of the quality of the lateral flow stick 110b, for example determine whether the milk sample could be applied to the lateral flow stick 110b, or not.

The method 400 in addition comprises performing 404 an action in relation to the lateral flow stick 110b, based on the quality of that lateral flow stick 110b as indicated by the detected 403 visual indication 180.

The action to be performed in relation to the lateral flow stick 110b may comprise applying a milk sample of milk extracted from an animal 301 to the lateral flow stick 110b when the detected 403 visual indication 180 of that lateral flow stick 110b allow the lateral flow stick 110b to be used for determining a biomarker value of the milk sample.

Also, or alternatively, the action to be performed in relation to the lateral flow stick 110b may comprise disallowing application of a milk sample of milk extracted from the animal 301 to the lateral flow stick 110b when the detected 403 visual indication 180 of that lateral flow stick 110b does not allow the lateral flow stick 110b to be used for determining the biomarker value of the milk sample.

The action to be performed in relation to the lateral flow stick 110b, in some embodiments, when the detected 403 visual indication 180 of that lateral flow stick 110b does not allow the lateral flow stick 110b to be used for determining the biomarker value, may comprise forwarding the carrier 120 in the Y-direction to a subsequent lateral flow stick 110c on the carrier 120, for repeating the method steps 401-404 on the subsequent lateral flow stick 110c.

The action to be performed in relation to the lateral flow stick 110b when the detected 403 visual indication 180 of that lateral flow stick 110b does not allow the lateral flow stick 110b to be used for determining the biomarker value, may alternatively comprise adjusting the speed when forwarding the carrier 120 in the Y-direction to the subsequent lateral flow stick 110c.

The measured biomarker may be e.g. progesterone, glycoprotein, oestrogen and/or Gonadatropin-Releasing Hormones, or any other similar biomarker associated with reproduction or health of the animal 301, in different embodiments. However, the measured biomarker may in some embodiments comprise LDH (Lactate Dehydrogenase), BHB (Beta-HydroxyButyrate), and/or urea; or other biomarker related to status of the animal 301. In some embodiments, a plurality of the above enumerated biomarkers may be measured.

The lateral flow sticks 110a, 110b, 110c may be arranged to indicate a biomarker in the milk sample of the animal 301 when a lateral flow test is performed. The lateral flow sticks 110a, 110b, 110c may comprise a sample pad onto which a milk sample may be applied. The lateral flow sticks 110a, 110b, 110c may also comprise a conjugate pad comprising conjugate which is dispersed into the milk sample when the milk sample is flowing from the sample pad through the conjugate pad. The lateral flow sticks 110a, 110b, 110c may furthermore comprise a porous membrane for receiving a capillary flow of the milk sample from the conjugate pad, thereby forwarding conjugate dispersed into the milk sample to a test line and a control line. The test line is prepared to change colour tint when exposed for milk comprising the biomarker while the control line is prepared to change colour tint when exposed for the milk sample, independently of presence of the biomarker in the milk sample.

The terminology used in the description of the embodiments as illustrated in the accompanying drawings is not intended to be limiting of the described arrangement 100 and/or method 400. Various changes, substitutions and/or alterations may be made, without departing from invention embodiments as defined by the appended claims.

As used herein, the term “and/or” comprises any and all combinations of one or more of the associated listed items. The term “or” as used herein, is to be interpreted as a mathematical OR, i.e., as an inclusive disjunction; not as a mathematical exclusive OR (XOR), unless expressly stated otherwise. In addition, the singular forms “a”, “an” and “the” are to be interpreted as “at least one”, thus also possibly comprising a plurality of entities of the same kind, unless expressly stated otherwise. It will be further understood that the terms “includes”, “comprises”, “including” and/or “comprising”, specifies the presence of stated features, actions, integers, steps, operations, elements, and/or components, but do not preclude the presence or addition of one or more other features, actions, integers, steps, operations, elements, components, and/or groups thereof. A single unit such as e.g. a processor may fulfil the functions of several items recited in the claims. The mere fact that certain measures or features are recited in mutually different dependent claims, illustrated in different figures or discussed in conjunction with different embodiments does not indicate that a combination of these measures or features cannot be used to advantage. A computer program may be stored/distributed on a suitable medium, such as an optical storage medium or a solid-state medium supplied together with or as part of other hardware but may also be distributed in other forms such as via Internet or other wired or wireless communication system.

1. An arrangement configured to determine quality of a lateral flow stick arranged on a carrier comprising a longitudinal extension in a Y-direction comprising a plurality of lateral flow sticks that are separated from each other at a distance (d) from each other in the Y-direction; wherein an elongated direction of each lateral flow stick extends in a substantially transverse direction in relation to the Y-direction; wherein the lateral flow sticks are configured for determining a biomarker value of milk extracted from an animal wherein the arrangement comprises:
a camera directed to capture an image of at least one of the lateral flow sticks out of the plurality of lateral flow sticks, after having been arranged on the carrier;
a marker configured to apply a visual indication on the lateral flow stick, or at the carrier, indicating a quality of that lateral flow stick;
a controller, communicatively connected to the camera and to the marker, wherein the controller is configured to
obtain an image of the at least one lateral flow stick and part of the carrier from the camera;
perform image processing of the obtained image;
determine quality of the at least one lateral flow stick based on the performed image processing; and
generate a command to the marker, to apply the visual indication indicating quality of the at least one lateral flow stick.

2. The arrangement according to claim 1, wherein the controller is configured to perform the image processing and estimate width (w) of the at least one lateral flow stick; and wherein the quality is related to the estimated width (w) of the at least one lateral flow stick.

3. The arrangement according to claim 1, wherein the controller is configured to perform the image processing by contour analysis of the at least one lateral flow stick in the image; and
determine an outer contour of the at least one lateral flow stick based on a contour analysis;
compare the determined outer contour of the at least one lateral flow stick with a reference contour; and wherein the quality is related to a difference between the determined outer contour of the at least one lateral flow stick and the reference contour.

4. The arrangement according to claim 1, wherein the controller is configured to, based on the image processing:
detect discolouration on the at least one lateral flow stick; and wherein the quality is related to colour and/or discolouration of the at least one lateral flow stick.

5. The arrangement according to claim 1, wherein the controller is configured to, based on the image processing:
determine whether the at least one lateral flow stick is arranged on the carrier in a predetermined way; and wherein the quality is related to the arrangement of the at least one lateral flow stick on the carrier in comparison with the predetermined way of arrangement.

6. The arrangement according to claim 1, wherein shape of the visual indication, number of visual indications and/or position of the visual indications at the carrier or the lateral flow stick, forms a carrier of information concerning the determined quality of the lateral flow stick.

7. The arrangement according to claim 1, wherein the marker is configured to apply the visual indication by punching a hole in the carrier.

8. The arrangement according to claim 1, wherein the marker is configured to apply the visual indication on a predetermined position on the carrier.

9. The arrangement according to claim 8, wherein the predetermined position for the visual indication is situated on the carrier in close vicinity of the lateral flow stick to which the quality is determined.

10. The arrangement according to claim 8, wherein the carrier is configured to be forwarded in the Y-direction, thereby sequentially placing one lateral flow stick at the time in a position where the camera is enabled to capture the image; and wherein the predetermined position for the visual indication of the lateral flow stick to which the quality is determined is situated in close vicinity of a previous lateral flow stick in the Y-direction.

11. A method at a farm, to perform an action in relation to a lateral flow stick on a carrier that comprises a plurality of lateral flow sticks, based on the quality of that lateral flow stick as indicated by a visual indication that has been provided to the carrier and/or the lateral flow stick by an arrangement according to claim 1; wherein the method comprises the steps of:
obtaining an image of a part of the carrier and/or the lateral flow stick;
performing image processing of the obtained image;
detecting the visual indication on the lateral flow stick, or on the carrier, indicating a quality of that lateral flow stick; and
performing an action in relation to the lateral flow stick, based on the quality of that lateral flow stick as indicated by the detected visual indication.

12. The method according to claim 11, wherein the action to be performed in relation to the lateral flow stick comprises applying a milk sample of milk extracted from an animal to the lateral flow stick when the detected visual indication of that lateral flow stick allow the lateral flow stick to be used for determining a biomarker value of the milk sample.

13. The method according to claim 11, wherein the action to be performed in relation to the lateral flow stick comprises disallowing application of a milk sample of milk extracted from an animal to the lateral flow stick when the detected visual indication of that lateral flow stick does not allow the lateral flow stick to be used for determining a biomarker value of the milk sample.

14. The method according to claim 13, wherein the carrier has a longitudinal extension in a Y-direction and arranged to be forwarded in the Y-direction, thereby sequentially placing one lateral flow stick at the time in a position where the application of the milk sample is enabled; wherein each one of the lateral flow sticks is individually arranged, separated with a minimum distance from any other lateral flow stick on the carrier; and wherein the action to be performed in relation to the lateral flow stick when the detected visual indication of that lateral flow stick does not allow the lateral flow stick to be used for determining the biomarker value comprises
forwarding the carrier in the Y-direction to a subsequent lateral flow stick for repeating the method steps 401-404 on the subsequent lateral flow stick.

15. The method according to claim 14, wherein the action to be performed in relation to the lateral flow stick when the detected visual indication of that lateral flow stick does not allow the lateral flow stick to be used for determining the biomarker value, comprises
adjusting the speed when forwarding the carrier in the Y-direction to the subsequent lateral flow stick.

16. The method according to claim 11, wherein the image is obtained from a part of the carrier comprising a predetermined position dedicated for the visual indication indicating quality of one particular lateral flow stick.
